(19) United States
(12) Patent Application Publication
(10) Pub. No.: US 2015/0037791 A1
(43) Pub. Date: Feb. 5, 2015

(54) METHODS AND KITS FOR MULTIPLEX AMPLIFICATION OF SHORT TANDEM REPEAT LOCI

(71) Applicant: LIFE TECHNOLOGIES CORPORATION, Carlsbad, CA (US)

(72) Inventors: Lori HENNESSY, San Mateo, CA (US); Dennis WANG, Dublin, CA (US)

(21) Appl. No.: 14/328,074

(22) Filed: Jul. 10, 2014

Related U.S. Application Data
(63) Continuation of application No. 13/291,976, filed on Nov. 8, 2011, now abandoned.
(60) Provisional application No. 61/526,195, filed on Aug. 22, 2011.
(60) Provisional application No. 61/413,946, filed on Nov. 15, 2010.

(51) Int. Cl. C12Q 1/68

(52) U.S. Cl. CPC C12Q 1/6827
USPC 435/6.11

(57) ABSTRACT

Compositions, methods and kits are disclosed for use in simultaneously amplifying at least 20 specific STR loci of genomic nucleic acid in a single multiplex reaction, as are methods and materials for use in the analysis of the products of such reactions. Included in the present invention are materials and methods for the simultaneous amplification of 23 and 24 specific loci in a single multiplex reaction, comprising the 13 CODIS loci, the Amelogenin locus, an InDel and at least six to ten additional STR loci, including methods, kits and materials for the analysis of these loci.

This application is a continuation of U.S. patent application Ser. No. 13/291,976 filed Nov. 8, 2011, which claims a priority benefit under 35 U.S.C. §119(e) from U.S. Patent Application No. 61/413,946 filed Nov. 15, 2010 and U.S. Patent Application No. 61/526,195 filed Aug. 22, 2011, which are incorporated herein by reference.

### FIELD

The present teachings relate to compositions, methods and kits for short tandem repeat (STR) loci when performing multiplex analysis.

### INTRODUCTION

The present teachings are generally directed to the arrangement and detection of genetic markers in a genomic system. In various embodiments, multiple distinct polymorphic genetic loci are simultaneously amplified in one multiplex reaction in order to determine the alleles of each locus. The polymorphic genetic loci analyzed may be short tandem repeat (STR) loci, insertion/deletion polymorphisms and single nucleotide polymorphisms (SNPs) and can also include mini-STRs which produce amplicons of approximately 200 base pairs or fewer.

### SUMMARY

In accordance with the embodiments, there is disclosed a composition for genotyping nucleic acid from a sample wherein the nucleic acid from the sample is amplified with a plurality of amplification primer pairs to form a plurality of amplification products; wherein at least one of each of said primer pairs comprises one of at least five different labels; wherein each of said amplification products comprise a different STR marker yielding an STR marker amplification product. The STR marker amplification products are separated by a mobility-dependent separation method; wherein a first primer set labeled with a first label comprises at least three different STR marker amplification products selected from D3S1358, vWA, TPOX, D16S539, CSF1PO, DYS391, and D7S820; and a second primer set labeled with a second label comprises at least three different STR marker amplification products selected from D5S818, D21S11, D8S1179, and D18S51, Y InDel rs 2032678 and a sex-determination marker AMEL; a third primer set labeled with a third label comprises at least three different STR marker amplification products selected from D2S441, D19S433, TH01 and FGA and a fourth primer set labeled with a fourth label comprises at least three different STR marker amplification products D22S1045, D5S818, D8S1179, D13S317, D16S539, D2S1338, D7S820, D6S1043, and SE33; and a fifth primer set labeled with a fifth label comprises at least three different STR marker amplification products selected from D10S1248, D1S1656, D12S391, CSF1PO, D2S1338, and Penta E; and the genotype is then determined for the nucleic acid from the sample by identifying each allele(s) for each of said different STR marker amplification products.

In some embodiments, the present teachings provide a method for genotyping nucleic acid from a sample and a method wherein a set of loci of at least one DNA sample to be analyzed is co-amplified in a multiplex amplification reaction with a plurality of amplification primer pairs to form a plurality of amplification products in a mixture, wherein at least one of each of said primer pairs comprises one of at least five different labels, wherein each of said amplification products comprise a different STR marker yielding amplified alleles in an STR marker amplification product, wherein the set of loci comprises at least three loci containing STR markers selected from D3S1358, vWA, TPOX, D16S539, CSF1PO, DYS391, and D7S820 in a first labeled STR marker amplification product set; at least three loci containing STR markers selected from D5S818, D21S11, D8S1179, and D18S51, Y InDel rs 2032678 and a sex-determination marker AMEL in a second labeled STR marker amplification product set; at least three loci containing STR markers selected from D2S441, D19S433, TH01 and FGA in a third labeled STR marker amplification product set; at least three loci containing STR markers selected from D22S1045, D5S818, D8S1179, D13S317, D16S539, D2S1338, D7S820, D6S1043, and SE33 in a fourth labeled STR marker amplification product set; and at least three loci containing STR markers selected from D10S1248, D1S1656, D12S391, CSF1PO, D2S1338, and Penta E in a fifth labeled STR marker amplification product set and evaluating the amplified alleles as well as the sex-determination marker, InDel and STR marker amplification products mixture to determine the alleles present at each of the loci analyzed in the set of loci within the at least one DNA sample.

In some embodiments, the present teachings provide a method of simultaneously determining the alleles present in at least four STR loci from one or more DNA samples, comprising: selecting a set of at least four STR loci of the DNA sample to be analyzed which can be amplified together, wherein the at least four loci in the set are selected from the group of loci consisting of: an InDel, SE33, D5S818, D7S820, D16S539, D18S51, D19S433, D21S11, D2S1338, D3S1358, D8S1179, FGA, TH01, VWA, TPOX, D13S317, CSF1PO, D10S1248, D12S391, D1S1656, D22S1045, D6S1043, D2S1360, D3S1744, D4S2366, D5S2500, D6S474, D6S1043, D8S1132, D7S1517, D10S2325, D21S2055, D10S2325, D2S441, D10S1248, Penta E, Penta D, LPL, F13B, FESFPS, F13A01, Penta C, DYS391, D12S391, AMEL, DYS19, DYS385, DYS389-I DYS389-II, DYS390, DYS392, DYS393, DYS437, DYS438, DYS439, and SPY; co-amplifying the loci in the set in a multiplex amplification reaction, wherein the product of the reaction is a mixture of amplified alleles from each of the co-amplified loci in the set; and evaluating the amplified alleles in the mixture to determine the alleles present at each of the loci analyzed in the set within the DNA sample. In some embodiments the InDel is rs 2032678. In some embodiments of a method a set of at least four loci are co-amplified, wherein the set of four loci is selected from the group of sets of loci consisting of: SE33, D5S818, D7S820, AMEL; SE33, D22S1045, AMEL,

### YS391; SE33, Penta E, YS391, AMEL; SE33, D12S391, YS391, AMEL; and D12S391, D2S13600, AMEL, SE33.

In some embodiments, the present teachings provide a kit comprising oligonucleotide primers for co-amplifying a set of loci of at least one DNA sample to be analyzed; wherein the set of loci can be co-amplified; wherein the primers are in one or more containers; and wherein the set of loci comprises the Amelogenin locus, the insertion/deletion (InDel) rs 2032678, the STR loci D16S539, D18S51, D19S433, D21S11, D2S1338, D3S1358, D8S1179, FGA TH01, vWA, TPOX, DS818, D7S820, D13S317, CSF1PO1PO, and at least one or more of the group consisting of the STR loci D16S539, D18S51, D19S433, D21S11, D3S1358, D8S1179, FGA TH01, VWA, TPOX, DS818, D7S820, D13S317, CSF1PO, and at least one or more of the group consisting of the STR loci D2S1338, D10S1248, D12S391, D1S1656, D22S1045, D6S1043, SE33, Penta D, Penta E, D2S1360, D3S1744, D4S2366, D5S2500, D6S474, D8S1132, D7S1517, D1052325, D21S2055, D22S1045, D21S2055, D6S1043, D2S441, DYS19, DYS385, DYS389-I DYS389-II, DYS390, DYS392, DYS393, DYS437, DYS438, DYS439, and the SPY locus.

In the following description, certain aspects and embodiments will become evident. It should be understood that a given embodiment need not have all aspects and features described herein. It should be understood that these aspects and embodiments are merely exemplary and explanatory and are not restrictive of the invention.

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain teachings.

Matching DNA profiles produced from existing commercial STR assays with improved STR assays provides continuity and comparability of the DNA profiles within and between databases. The increase in loci reduces the likelihood of adventitious matches, increases international data overlap and compatibility and increases discrimination power useful for missing person cases. Adding additional loci for improved discrimination and identification for both database and casework samples also necessitate continuity and comparability with existing DNA profiles while improving efficiency and simplifying workflows suitable for automation. The occurrence of allelic dropout in new STR assays can make DNA profile matching within and between databases difficult or imprecise. Thus, careful design of new assays such that all potential amplification products are detected in as large a portion of the population as possible remains an ongoing concern when developing new STR assays. Therefore, there exists a need in the art, to improve DNA-based technologies based on the discovery of new sample processing, preclusion of known causes of allelic dropout and verification of gender results. These and other features of the present teachings are set fourth herein.

### BRIEF DESCRIPTION OF THE DRAWINGS

The skilled artisan will understand that the figures, described below, are for illustration purposes only. The figures are not intended to limit the scope of the present teachings in any way.

FIG. 1 demonstrates the relative size ranges of the amplicons (in base pairs) as produced by multiplex amplification of twenty STR loci and the Amelogenin sex determination locus (Amel), as described in Example I.

FIG. 2 demonstrates the relative size ranges of the amplicons (in base pairs) as produced by multiplex amplification of twenty-three STR loci, 1 indel marker and the Amelogenin sex determination locus (Amel) as described in Example II.

FIG. 3 demonstrates the relative size ranges of the amplicons (in base pairs) as produced by multiplex amplification of twenty-two STR loci, 1 InDel marker and the Amelogenin sex determination locus (Amel) by direct amplification, as described in Example III.

### DETAILED DESCRIPTION

For the purposes of interpreting of this specification, the following definitions will apply and whenever appropriate, terms used in the singular will also include the plural and vice versa. In the event that any definition set fourth below conflicts with the usage of that word in any other document, including any document incorporated herein by reference, the definition set fourth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

The section headings used herein are for organizational purposes only and are not to be construed as limiting the described subject matter in any way. All literature cited in this specification, including but not limited to, patents, patent applications, articles, books, and treatises are expressly incorporated by reference in their entirety for any purpose. In the event that any of the incorporated literature contradicts any term defined herein, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The practice of the present invention may employ conventional techniques and descriptions of organic chemistry, polymer technology, molecular biology (including recombinant techniques), cell biology, biochemistry, and immunology, which are within the skill of the art. Such conventional techniques include oligonucleotide synthesis, hybridization, extension reaction, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press, 1989), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes

As used herein, “DNA” refers to deoxyribonucleic acid in its various forms as understood in the art, such as genomic DNA, cDNA, isolated nucleic acid molecules, vector DNA, and chromosomal DNA. “Nucleic acid” refers to DNA or RNA (ribonucleic acid) in any form. As used herein, the term “isolated nucleic acid molecule” refers to a nucleic acid molecule (DNA or RNA) that has been removed from its native environment. Some examples of isolated nucleic acid molecules are recombinant DNA molecules contained in a vector, recombinant DNA molecules maintained in a heterologous host cell, partially or substantially purified nucleic acid molecules, and synthetic DNA molecules. An “isolated” nucleic acid can be free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

“Short tandem repeat” or “STR” loci refer to regions of genomic DNA which contain short, repetitive sequence elements. The sequence elements that are repeated are not limited to but are generally three to seven base pairs in length. Each sequence element is repeated at least once within an STR and is referred to herein as a “repeat unit.” The term STR also encompasses a region of genomic DNA wherein more than a single repeat unit is repeated in tandem or with intervening bases, provided that at least one of the sequences is repeated at least two times in tandem.

“Polymorphic short tandem repeat loci” refers to STR loci in which the number of repetitive sequence elements (and net length of the sequence) in a particular region of genomic DNA varies from allele to allele, and from individual to individual.

As used herein, “allelic ladder” refers to a standard size marker consisting of amplified alleles from the locus. “Allele” refers to a genetic variation associated with a segment of DNA; i.e., one of two or more alternate forms of a DNA sequence occupying the same locus.

“Biochemical nomenclature” refers to the standard biochemical nomenclature as used herein, in which the nucleotide bases are designated as adenine (A), thymine (T), guanine (G), and cytosine (C). Corresponding nucleotides are, for example, deoxyguanosine-5′-triphosphate (dGTP).

“DNA polymorphism” refers to the condition in which two or more different nucleotide sequences in a DNA sequence coexist in the same interbreeding population.

As used herein, the term “kit” refers to any delivery system for delivering materials. In the context of reaction assays, such delivery systems include systems that allow for the storage, transport, or delivery of reaction reagents (e.g., oligonucleotides, enzymes, primer set(s), etc. in the appropriate containers) and/or supporting materials (e.g., buffers, written instructions for performing the assay etc.) from one location to another. For example, kits can include one or more enclosures (e.g., boxes) containing the relevant reaction reagents and/or supporting materials. As used herein, the term “fragmented kit” refers to a delivery system comprising two or more separate containers that each contains a subportion of the total kit components. The containers may be delivered to the intended recipient together or separately. For example, a first container may contain an enzyme for use in an assay, while a second container contains oligonucleotides. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

“Locus” or “genetic locus” refers to a specific physical position on a chromosome. Alleles of a locus are located at identical sites on homologous chromosomes.

“Locus-specific primer” refers to a primer that specifically hybridizes with a portion of the stated locus or its complementary strand, at least for one allele of the locus, and does not hybridize efficiently with other DNA sequences under the conditions used in the amplification method.

“Polymerase chain reaction” or “PCR” refers to a technique in which repetitive cycles of denaturation, annealing with a primer, and extension with a DNA polymerase enzyme are used to amplify the number of copies of a target DNA sequence by approximately 10⁶ times or more. The PCR process for amplifying nucleic acids is covered by U.S. Pat. Nos. 4,683,195 and 4,683,202, which are herein incorporated in their entirety by reference for a description of the process. The reaction conditions for any PCR comprise the chemical components of the reaction and their concentrations, the temperatures used in the reaction cycles, the number of cycles of the reaction, and the durations of the stages of the reaction cycles.

As used herein, “amplify” refers to the process of enzymatically increasing the amount of a specific nucleotide sequence. This amplification is not limited to but is generally accomplished by PCR. As used herein, “denaturation” refers to the separation of two complementary nucleotide strands from an annealed state. Denaturation can be induced by a number of factors, such as, for example, ionic strength of the buffer, temperature, or chemicals that disrupt base pairing interactions. As used herein, “annealing” refers to the specific interaction between strands of nucleotides wherein the strands bind to one another substantially based on complementarity between the strands as determined by Watson-Crick base pairing. It is not necessary that complementarity be 100% for annealing to occur. As used herein, “extension” refers to the amplification cycle after the primer oligonucleotide and target nucleic acid have annealed, wherein the polymerase enzyme affects primer extension into the appropriately sized fragments using the target nucleic acid as replicative template.

“Primer” refers to a single-stranded oligonucleotide or DNA fragment which hybridizes with a DNA strand of a locus in such a manner that the 3′ terminus of the primer can act as a site of polymerization and extension using a DNA polymerase enzyme. “Primer pair” refers to two primers comprising a primer 1 that hybridizes to a single strand at one end of the DNA sequence to be amplified, and a primer 2 that hybridizes with the other end on the complementary strand of the DNA sequence to be amplified. A primer pair can also include a primer 3 which is a degenerate primer with respect to either primer 1 or primer 2. “Primer site” refers to the area of the target DNA to which a primer hybridizes.

“Genetic markers” are generally a set of polymorphic loci having alleles in genomic DNA with characteristics of interest for analysis, such as DNA typing, in which individuals are differentiated based on variations in their DNA. Most DNA typing methods are designed to detect and analyze differences in the length and/or sequence of one or more regions of DNA markers known to appear in at least two different forms, or alleles, in a population. Such variation is referred to as “polymorphism,” and any region of DNA in which such a variation occurs is referred to as a “polymorphic locus.” One possible method of performing DNA typing involves the joining of PCR amplification technology (K B Mullis, U.S. Pat. No. 4,683,202) with the analysis of length variation polymorphisms. PCR traditionally could only be used to amplify relatively small DNA segments reliably; i.e., only amplifying DNA segments under 3,000 bases in length (M. Ponce and L. Micol (1992), NAR 20(3):623; R. Decorte et al. (1990), DNA CELL BIOL. 9(6):461 469). Short tandem repeats (STRs), minisatellites and variable number of tandem repeats (VNTRs) are some examples of length variation polymorphisms. DNA segments containing minisatellites or VNTRs are generally too long to be amplified reliably by PCR. By contrast STRs, containing repeat units of approximately three to seven nucleotides, are short enough to be useful as genetic markers in PCR applications, because amplification protocols can be designed to produce smaller products than are possible from the other variable length regions of DNA.

It is often desirable to amplify and detect multiple loci simultaneously in a single amplification reaction and separation process. Such compositions simultaneously targeting several loci for analysis are called “multiplex” systems. Several such systems containing multiple STR loci have been described. See, e.g., AMPFLSTR® SGMPLUS™ PCR AMPLIFICATION KIT USER'S MANUAL, Applied Biosystems, pp. i-x and 1-1 to 1-16 (2001); AMPFLSTR® IDENTIFILER® PCR AMPLIFICATION KIT USER'S MANUAL, Applied Biosystems, pp. i-x and 1-1 to 1-10 (2001); J W Schumm et al., U.S. Pat. No. 7,008,771.

The governments of several countries maintain databases of DNA typing information. The National DNA Database of the United Kingdom (NDNAD) is the largest such database, with the DNA profiles of approximately 2.7 million people. H. Wallace (2006), EMBO REPORTS 7:S26-S30 (citing Home Office, 2006). Since 1999, the DNA profiles in the NDNAD have been based on the SGMplus® system, developed by Applied Biosystems. Id. A recurring problem in DNA profiling systems is how to identify individuals when their DNA samples are degraded. A number of studies have been performed in labs in Europe and the United States to compare conventional STRs (amplicons which range in size from about 100 to about 450 base pairs) with mini-STRs (amplicons of 200 base pairs or fewer) as genetic markers in analyzing degraded DNA samples. See, e.g., L A Dixon et al. (2006), FORENSIC SCI. INT. 164(1):33-44. The results indicate that the chances of obtaining successful results from the analysis of degraded DNA samples improves with smaller sized amplicons, such as are obtained from mini-STR loci. Id.; M D Coble and J M Butler (2005), J. FORENSIC SCI. 50(1):43-53. The European Network of Forensic Science Institutes (ENFSI) and European DNA Profiling (EDNAP) group agreed that multiplex PCR systems for DNA typing should be re-engineered to enable small amplicon detection, and that standardization of profiling systems within Europe should take account of mini-STRs. P. Gill et al. (2006), FORENSIC SCI. INT. 156(2-3):242-244. The present teachings relate to the simultaneous analysis of multiple length variation polymorphisms in a single reaction. Various embodiments of the present teachings incorporate mini-STR loci in multiplex amplification systems. These systems are amenable to various applications, including their use in DNA typing.

The methods of the present teachings contemplate selecting an appropriate set of loci, primers, and amplification protocols to generate amplified alleles (amplicons) from multiple co-amplified loci, which amplicons can be designed so as not to overlap in size, and/or can be labeled in such a way as to enable one to differentiate between alleles from different loci which do overlap in size. In addition, these methods contemplate the selection of multiple STR loci which are compatible for use with a single amplification protocol. In addition, these multiple STR loci can be amplified in a single amplification protocol in under 40 minutes, under 35 minutes or under 30 minutes or less. The specific combinations of loci described herein are unique in this application. Also contemplated in the methods is the ability to replace one locus directly for another locus, including but not limited to substituting D6S1043 for SE33. In various embodiments of the present teachings a co-amplification of at least 20, of at least 21, of at least 22, and of at least 23 or more STR loci is taught, which comprises at least six, at least seven, or at least eight mini-STR loci with a maximum amplicon size of less than approximately 200 base pairs. Also included is a sex-determination marker, Amelogenin (AMEL). Also included is an additional Y-marker to provide gender confirmation in instances of Amelogenin dropout and minimize the occurrence of a double deletion event. Also included is at least one, at least two, at least three, and at least four insertion/deletion (indel) polymorphic marker(s).

In some embodiments, the inclusion of degenerate primers for D3S1358, D18S51, D19S433, TH01, D5S818, VWA, FGA, and SE33 were done to minimize false homozygosity that has been reported in the literature.

Successful combinations in addition to those disclosed herein can be generated by, for example, trial and error of locus combinations, by selection of primer pair sequences, and by adjustment of primer concentrations to identify equilibrium in which all loci for analysis can be amplified. Once the methods and materials of these teachings are disclosed, various methods of selecting loci, primer pairs, and amplification techniques for use in the methods and kits of these teachings are likely to be suggested to one skilled in the art. All such methods are intended to be within the scope of the appended claims.

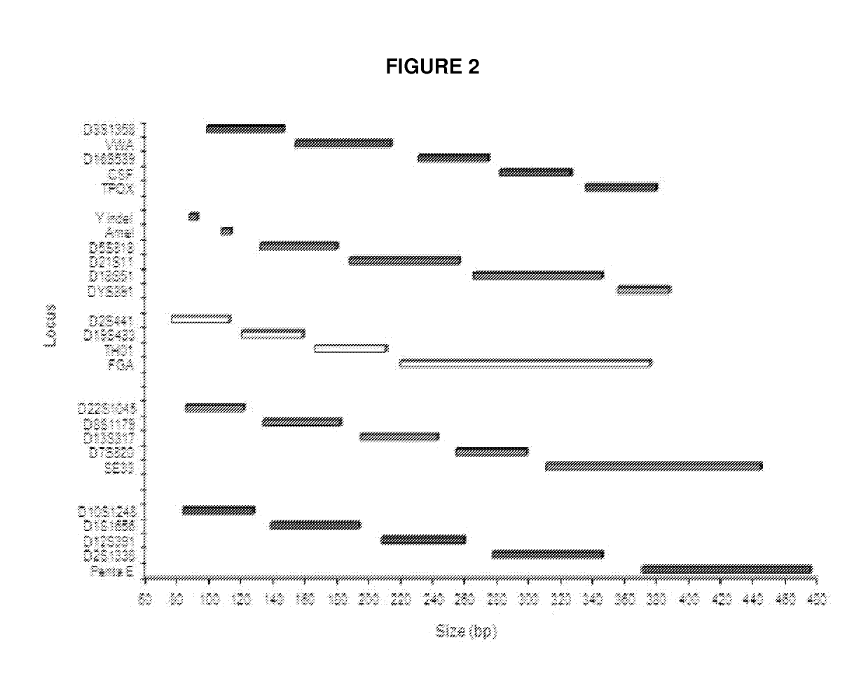

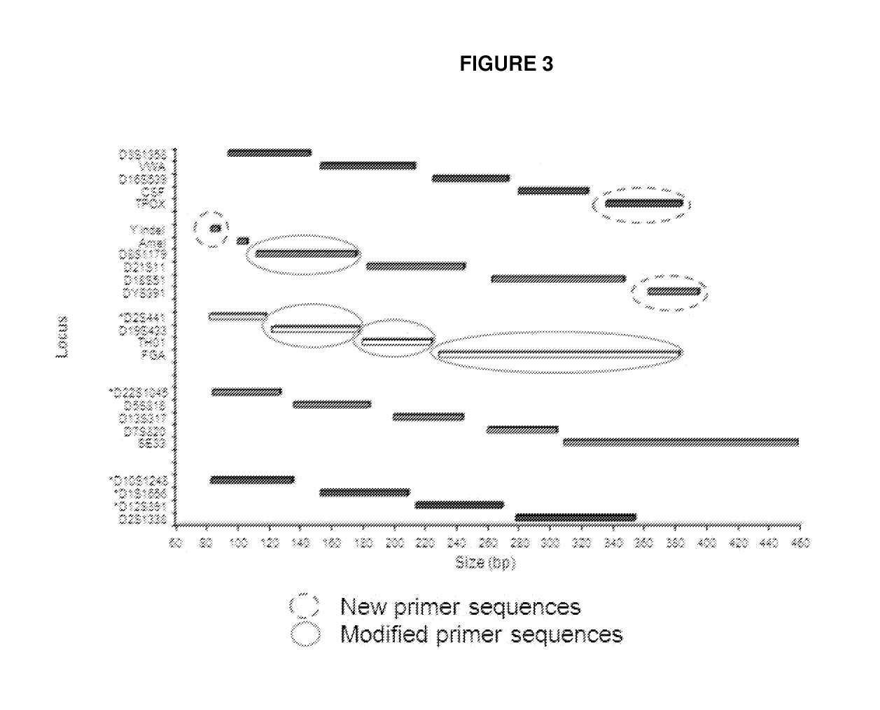

Practice of the methods of the present teaching may begin with selection of a set of at least eleven STR loci comprising D16S539, D18S51, D19S433, D21S11, D2S1338, D3S1358, D8S1179, FGA (also known as FIBRA), TH01 (also known as TC11), VWA, TPOX, D5S818, D7S820, D13S317, CSF (also known as CSF1PO), and at least one of the STR loci D10S1248, D12S391, D1S1656, D22S1045, D6S1043, SE33, D2S1360, D3S1744, D4S2366, D5S2500, D6S474, D6S1043, D8S1132, D7S1517, D10S2325, D21S2055, D10S2325, D2S441, D10S1248, Penta E, Penta D, LPL, F13B, FESFPS, F13A01, Penta C, DYS391, and D12S391, all of which can be co-amplified in a single multiplex amplification reaction. Other loci besides or in addition to the listed loci may be included in the multiplex amplification reaction, including the insertion/deletion (Indel) rs 2032678, and a gender loci selected from AMEL DYS19, DYS385, DYS389-I DYS389-II, DYS390, DYS392, DYS393, DYS437, DYS438, DYS439, and SPY. Possible methods for selecting the loci and oligonucleotide primers to amplify the loci in the multiplex amplification reaction of the present teachings are described herein and illustrated in the Examples below. Figures representing a 6-dye multiplex emission spectra are presented in previously filed US application U.S. Ser. No. 12/261,506, filed Oct. 30, 2008 and incorporated by reference herein and are illustrated in FIGS. 2 and 3.

Any of a number of different techniques can be used to select the set of loci for use according to the present teachings. Once a multiplex containing the at least eleven STR loci is developed, it can be used as a core to create multiplexes containing more than these eleven loci, and containing loci other than STR loci; for example, an indel or a sex determination locus or a Y STR locus. New combinations of more than eleven loci can thus be created comprising the first eleven STR loci.

Regardless of what methods may be used to select the loci analyzed by the methods of the present teaching, the loci selected for multiplex analysis in various embodiments share one or more of the following characteristics: (1) they produce sufficient amplification products to allow allelic evaluation of the DNA; (2) they generate few, if any, artifacts during the multiplex amplification step due to incorporation of additional bases during the extension of a valid target locus or the production of non-specific amplicons; and (3) they generate few, if any, artifacts due to premature termination of amplification reactions by a polymerase. See, e.g., J W Schumm et al. (1993), FOURTH INTERNATIONAL SYMPOSIUM ON HUMAN IDENTIFICATION, pp. 177-187, Promega Corp.

The terms for the particular STR loci as used herein refer to the names assigned to these loci as they are known in the art. The loci are identified, for example, in the various references and by the various accession numbers in the list that follows, all of which are incorporated herein by reference in their entirety. The list of references that follows is merely intended to be exemplary of sources of locus information. The information regarding the DNA regions comprising these loci and contemplated for target amplification are publicly available and easily found by consulting the following or other references and/or accession numbers. Where appropriate, the current Accession Number as of time of filing is presented, as provided by GenBank® (National Center for Biotechnology Information, Bethesda, Md.). See, e.g., for the locus D3S1358, H. Li et al. (1993), HUM. MOL. GENET. 2:1327; for D12S391, M V Lareu et al. (1996), GENE 182:151-153; for D18S51, R E Staub et al. (1993), GENOMICS 15:48-56; for D21S11, V. Sharma and M. Litt (1992), Hum. MOL. GENET. 1:67; for FGA (FIBRA), K A Mills et al. (1992), HUM. MOL. GENET. 1:779; for TH01, A. Edwards (1991), AM. J. HUM. GENET. 49:746-756 and M H Polymeropoulos et al. (1991), NUCLEIC ACIDS RES. 19:3753; for VWA (vWF), C P Kimpton et al. (1992), HUM. MOL. GENET. 1:287; for D10S1248, M D Coble and J M Butler (2005), J. FORENSIC SCI. 50(1):43-53; for D16S539, J. Murray et al. (1995), unpublished, Cooperative Human Linkage Center, Accession Number G07925; for D2S1338, J. Murray et al. (1995), unpublished, Cooperative Human Linkage Center, Accession Number G08202 and Watson et al. in PROGRESS IN FORENSIC GENETICS 7: PROCEEDINGS OF THE 17^{TH} INT'L ISFH CONGRESS, OSLO, 2-6 Sep. 1997, B. Olaisen et al., eds., pp. 192-194 (Elsevier, Amsterdam); for D8S1179, J. Murray et al. (1995), unpublished, Cooperative Human Linkage Center, Accession Number G08710, and N J Oldroyd et al. (1995), ELECTROPHORESIS 16:334-337; for D22S1045, J. Murray et al. (1995), unpublished, Cooperative Human Linkage Center, Accession Number G08085; for D19S433, J. Murray et al. (1995), unpublished, Cooperative Human Linkage Center, Accession Number G08036, and M V Lareu et al. (1997), in PROGRESS IN FORENSIC GENETICS 7: PROCEEDINGS OF THE 17^{TH} INT'L ISFH CONGRESS, OSLO, 2-6 Sep. 1997, B. Olaisen et al., eds., pp. 192-200, Elsevier, Amsterdam; for D2S441, J. Murray et al. (1995), unpublished, Cooperative Human Linkage Center, Accession Number G08184; for D1S1656, J. Murray et al. (1995), unpublished, Cooperative Human Linkage Center, Accession Number G07820. The STRbase from NIST also provides detailed information on STR loci, http://www.cstl.nist.gov/div831/strbase/.

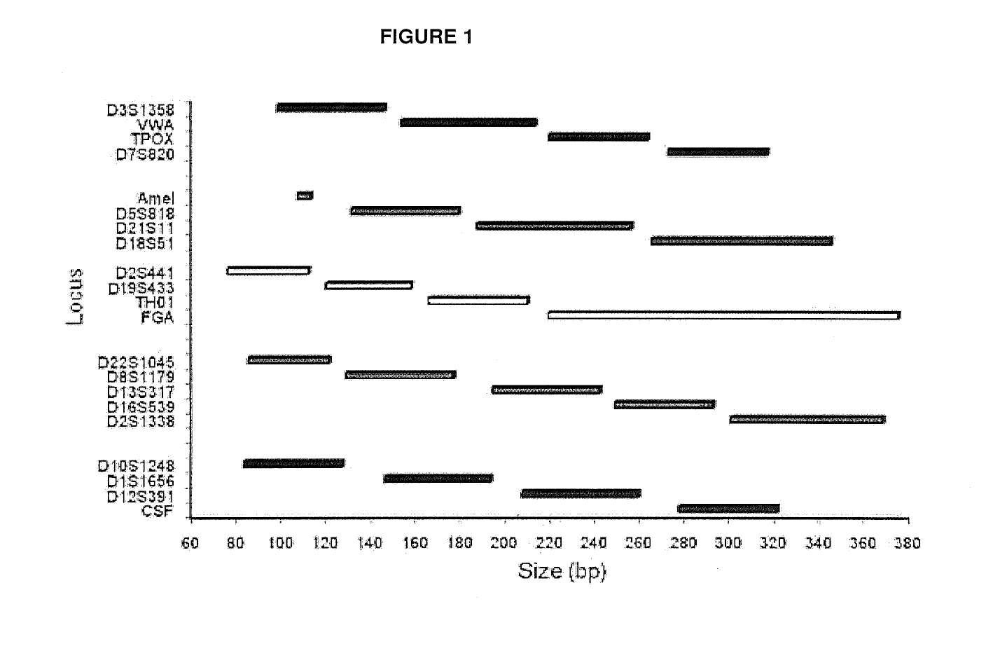

Amplification of mini-STRs (loci of fewer than approximately 200 base pairs) allows for the profiling analysis of highly degraded DNA, as is demonstrated in M D Coble (2005), J. FORENSIC SCI. 50(1):43-53, which is incorporated by reference herein. FIG. 1 demonstrates the locus size ranges for a multiplex of 20 loci described above, plus the Amelogenin locus for size determination. As can be seen in FIG. 1, eight of the loci identified in the preceding list comprise such mini-STR loci: D10S1248, VWA, D8S1179, D22S1045, D19S433, D2S441, D3S1358, D5S818, D12S391, and D1S1656. Table 1 (see U.S. Patent Application No. 61/413,946, filed Nov. 15, 2010 and Patent Application No. 61/526,195, filed Aug. 22, 2011 for Table 1) also provides loci that can be considered mini-STR loci depending on the positioning of the primers used to amplify the STR marker within a primer amplification set.

The set of loci selected for co-amplification and analysis according to these teachings can comprise at least one locus in addition to the at least eleven STR loci. The additional locus can comprise an STR or other sequence polymorphism, or any other feature, for example, which identifies a particular characteristic to separate the DNA of one individual from the DNA of other individuals in the population. The additional locus can also be one which identifies the sex of the source of the DNA sample analyzed. When the DNA sample is human genomic DNA, a sex-identifying locus such as the Amelogenin locus can be selected for co-amplification and analysis according to the present methods. The Amelogenin locus is identified by GenBank as HUMAMELY (when used to identify a locus on the Y chromosome as present in male DNA) or as HUMAMELX (when used to identify a locus on the X chromosome as present in male or female DNA).

Once a set of loci for co-amplification in a single multiplex reaction is identified, one can determine primers suitable for co-amplifying each locus in the set. Oligonucleotide primers may be added to the reaction mix and serve to demarcate the 5′ and 3′ ends of an amplified DNA fragment. One oligonucleotide primer anneals to the sense (+) strand of the denatured template DNA, and the other oligonucleotide primer anneals to the antisense (−) strand of the denatured template DNA. Typically, oligonucleotide primers may be approximately 12-25 nucleotides in length, but their size may vary considerably depending on such parameters as, for example, the base composition of the template sequence to be amplified, amplification reaction conditions, etc. The specific length of the primer is not essential to the operation of these teachings. Oligonucleotide primers can be designed to anneal to specific portions of DNA that flank a locus of interest, so as to specifically amplify the portion of DNA between the primer-complementary sites.

Oligonucleotide primers may comprise adenosine, thymidine, guanosine, and cytidine, as well as uracil, nucleoside analogs (for example, but not limited to, inosine, locked nucleic acids (LNA), non-nucleotide linkers, peptide nucleic acids (PNA) and phosporamidites) and nucleosides containing or conjugated to chemical moieties such as radionuclides (e.g., ³²P and ³⁵S), fluorescent molecules, minor groove binders (MGBs), or any other nucleoside conjugates known in the art.

Generally, oligonucleotide primers can be chemically synthesized. Primer design and selection is a routine procedure in PCR optimization. One of ordinary skill in the art can easily design specific primers to amplify a target locus of interest, or obtain primer sets from the references listed herein. All of these primers are within the scope of the present teachings.

Care should be taken in selecting the primer sequences used in the multiplex reaction. Inappropriate selection of primers may produce undesirable effects such as a lack of amplification, amplification at one site or multiple sites besides the intended target locus, primer-dimer formation, undesirable interactions between primers for different loci, production of amplicons from alleles of one locus which overlap (e.g., in size) with alleles from another locus, or the need for amplification conditions or protocols particularly suited for each of the different loci, which conditions/protocols are incompatible in a single multiplex system. Primers can be developed and selected for use in the multiplex systems of this teaching by, for example, employing a re-iterative process of multiplex optimization that is well familiar to one of ordinary skill in the art: selecting primer sequences, mixing the primers for co-amplification of the selected loci, co-amplifying the loci, then separating and detecting the amplified products to determine effectiveness of the primers in amplification.

As an example of primer selection, individual primers and primer pairs, identified in the references cited herein, provided in the Examples, or described in other references, which are useful in amplifying any of the above listed loci may be selected to amplify and analyze the STR loci according to the present teachings. As another example, primers can be selected by the use of any of various software programs available and known in the art for developing amplification and/or multiplex systems. See, e.g., Primer Express® software (Applied Biosystems, Foster City, Calif.). In the example of the use of software programs, sequence information from the region of the locus of interest can be imported into the software. The software then uses various algorithms to select primers that best meet the user's specifications.

Initially, this primer selection process may produce any of the undesirable effects in amplification described above, or an imbalance of amplification product, with greater product yield for some loci than for others because of greater binding strength between some primers and their respective targets than other primers, for example resulting in preferred annealing and amplification for some loci. Or, the primers may generate amplification products which do not represent the target loci alleles themselves; i.e., non-specific amplification product may be generated. These extraneous products resulting from poor primer design may be due, for example, to annealing of the primer with non-target regions of sample DNA, or even with other primers, followed by amplification subsequent to annealing.

When imbalanced or non-specific amplification products are present in the multiplex systems during primer selection, individual primers can be taken from the total multiplex set and used in an amplification with primers from the same or other loci to identify which primers contribute to the amplification imbalance or artifacts. Once two primers which generate one or more of the artifacts or imbalance are identified, one or both contributors can be modified and retested, either alone in a pair, or in the multiplex system (or a subset of the multiplex system). This process may be repeated until product evaluation results in amplified alleles with no or an acceptable level of amplification artifacts in the multiplex system.

The optimization of primer concentration can be performed either before or after determination of the final primer sequences, but most often may be performed after primer selection. Generally, increasing the concentration of primers for any particular locus increases the amount of product generated for that locus. However, primer concentration optimization is also a re-iterative process because, for example, increasing product yield from one locus may decrease the yield from another locus or other loci. Furthermore, primers may interact with each other, which may directly affect the yield of amplification product from various loci. In sum, a linear increase in concentration of a specific primer set does not necessarily equate with a linear increase in amplification product yield for the corresponding locus. Reference is made to M J Simons, U.S. Pat. No. 5,192,659, for a more detailed description of locus-specific primers, the teaching of which is incorporated herein by reference in its entirety.

Locus-to-locus amplification product balance in a multiplex reaction may also be affected by a number of parameters of the amplification protocol, such as, for example, the amount of template (sample DNA) input, the number of amplification cycles used, the annealing temperature of the thermal cycling protocol, and the inclusion or exclusion of an extra extension step at the end of the cycling process. An absolutely even balance in amplification product yield across all alleles and loci, although theoretically desirable, is generally not achieved in practice.

The process of determining the loci comprising the multiplex system and the development of the reaction conditions of this system can also be a re-iterative process. That is, one can first develop a multiplex system for a small number of loci, this system being free or nearly free of amplification artifacts and product imbalance. Primers of this system can then be combined with primers for another locus or several additional loci desired for analysis. This expanded primer combination may or may not produce amplification artifacts or imbalanced product yield. In turn, some loci may be removed from the system, and/or new loci can be introduced and evaluated.

One or more of the re-iterative selection processes described above can be repeated until a complete set of primers is identified, which can be used to co-amplify the at least eleven loci selected for co-amplification as described above, comprising the STR loci D5S818, VWA, D16S539, D2S1338, D8S1179, D21S11, D18S51, D19S433, TH01, FGA, CSF, D3S1358, and one or more of D10S1248, D12S391, D1S1656, D22S1045, D6S1043, SE33, D2S1360, D3S1744, D4S2366, D5S2500, D6S474, D6S1043, D7S820, D13S317, D10S1248, D2S441, D8S1132, D7S1517, D10S2325, D2152055, D10S2325, D2S441, TPOX, Penta E, Penta D, LPL, F13B, FESFPS, F13A01, Penta C, DYS391, and D12S391. Other loci besides or in addition to the listed loci may be included in the multiplex amplification reaction, including the insertion/deletion (Indel) rs 2032678, and a gender loci selected from AMEL DYS19, DYS385, DYS389-I DYS389-II, DYS390, DYS392, DYS393, DYS437, DYS438, DYS439, and SPY. It is understood that many different sets of primers can be developed to amplify a particular set of loci. Synthesis of the primers used in the present teachings can be conducted using any standard procedure for oligonucleotide synthesis known to those skilled in the art and/or commercially available. In various embodiments of the present teaching, at least 20 of these STR loci can be co-amplified in one multiplex amplification composition: VWA, D16S539, D2S1338, D8S1179, D21S11, D18S51, D19S433, TH01, FGA, D3S1358, CSF1PO, TPOX, D5S818, D7S820, D13S317, D1S1656, D10S1248, D22S1045, D2S441 and D12S391. In other embodiments of the present teaching, at least 21, at least 22, and at least 23 of the disclosed STR loci and others as listed in STRbase can be co-amplified in one multiplex amplification reaction, as well as and including the Amelogenin locus for sex determination of the source of the DNA sample. The addition of a Y specific STR marker can also enable verification of the Y contribution in a mixed sample. Table 1 lists exemplary configurations that can be used to format multiplex reactions for a five or a six-dye multiplex configuration (see U.S. Patent Application No. 61/413,946, filed Nov. 15, 2010 and Patent Application No. 61/526,195, filed Aug. 22, 2011 for Table 1).

Samples of genomic DNA can be prepared for use in the methods of the present teaching using any procedures for sample preparation that are compatible with the subsequent amplification of DNA. Many such procedures are known by those skilled in the art. Some examples are DNA purification by phenol extraction (J. Sambrook et al. (1989), in MOLECULAR CLONING: A LABORATORY MANUAL, SECOND EDITION, Cold Spring Harbor Laboratory Press, Cold Spring Harbor, N.Y., pp. 9.14-9.19), and partial purification by salt precipitation (S. Miller et al. (1988), NUCL. ACIDS RES. 16:1215) or chelex (P S Walsh et al. (1991), BIOTECHNIQUES 10:506-513; C T Comey et al. (1994), J. FORENSIC SCI. 39:1254) and the release of unpurified material using untreated blood (J. Burckhardt (1994), PCR METHODS AND APPLICATIONS 3:239-243; R B E McCabe (1991), PCR METHODS AND APPLICATIONS 1:99-106; B Y Nordvag (1992), BIOTECHNIQUES 12:4 pp. 490-492).

When the at least one DNA sample to be analyzed using the methods of this teaching is human genomic DNA, the DNA can be prepared from tissue samples such as, for example, one or more of blood, whole blood, a blood component, a tissue biopsy, lymph, bone, bone marrow, tooth, skin, for example skin cells contained in fingerprints, bone, tooth, amniotic fluid containing placental cells, and amniotic fluid containing fetal cells, chorionic villus, hair, skin, semen, anal secretions, feces, urine, vaginal secretions, perspiration, saliva, buccal swabs, various environmental samples (for example, agricultural, water, and soil), research samples generally, purified samples generally, and lysed cells, and/or mixtures of any of these or other tissues.

Optionally, DNA concentrations can be measured prior to use in the method of the present teaching, using any standard method of DNA quantification known to those skilled in the art. Such quantification methods include, for example, spectrophotometric measurement, as described by J. Sambrook et al. (1989), supra, Appendix E.5; or fluorometric methodology using a measurement technique such as that described by CF Brunk et al. (1979), ANAL. BIOCHEM. 92: 497-500. DNA concentration can be measured by comparison of the amount of hybridization of DNA standards with a human-specific probe such as that described by J S Waye et al. (1991), J. FORENSIC SCI. 36:1198-1203 (1991). Use of too much template DNA in the amplification reactions may produce amplification artifacts, which would not represent true alleles.

Samples containing blood or buccal samples can also be processed directly from FTA® paper (Whatman Inc., Piscataway, N.J.), Bode Buccal Collector, or swabs., Examples of swabs include but are not limited to, Copan 4N6 Forensic Flocked Swab (Copan, P/N 3520CS01, Murrieta, Calif.), Omi Swab (Whatman Inc., P/N 10005) and Puritan Cotton Swab (Puritan, P/N 25-806 1WC EC, various medical suppliers).

Once a sample of genomic DNA is prepared, the target loci can be co-amplified in the multiplex amplification step of the present teaching. Any of a number of different amplification methods can be used to amplify the loci, such as, for example, PCR (R K Saiki et al. (1985), SCIENCE 230: 1350-1354), transcription based amplification (D Y Kwoh and T J Kwoh (1990), AMERICAN BIOTECHNOLOGY LABORATORY, October, 1990) and strand displacement amplification (SDA) (G T Walker et al. (1992), PROC. NATL. ACAD. SCI., U.S.A. 89: 392-396). In some embodiments of the present teaching, multiplex amplification can be effected via PCR, in which the DNA sample is subjected to amplification using primer pairs specific to each locus in the multiplex.

The chemical components of a standard PCR generally comprise a solvent, DNA polymerase, deoxyribonucleoside triphosphates (“dNTPs”), oligonucleotide primers, a divalent metal ion, and a DNA sample expected to contain the target(s) for PCR amplification. Water can generally be used as the solvent for PCR, typically comprising a buffering agent and non-buffering salts such as KCl. The buffering agent can be any buffer known in the art, such as, but not limited to, Tris-HCl, and can be varied by routine experimentation to optimize PCR results. Persons of ordinary skill in the art are readily able to determine optimal buffering conditions. PCR buffers can be optimized depending on the particular enzyme used for amplification.

Divalent metal ions can often be advantageous to allow the polymerase to function efficiently. For example, the magnesium ion is one which allows certain DNA polymerases to function effectively. Typically MgCl₂ or MgSO₄ can be added to reaction buffers to supply the optimum magnesium ion concentration. The magnesium ion concentration required for optimal PCR amplification may depend on the specific set of primers and template used. Thus, the amount of magnesium salt added to achieve optimal amplification is often determined empirically, and is a routine practice in the art. Generally, the concentration of magnesium ion for optimal PCR can vary between about 1 and about 10 mM. A typical range of magnesium ion concentration in PCR can be between about 1.0 and about 4.0 mM, varying around a midpoint of about 2.5 mM. Alternatively, the divalent ion manganese can be used, for example in the form of manganese dioxide (MnO₂), titrated to a concentration appropriate for optimal polymerase activity, easily determined by one of skill in the art using standard laboratory procedures.

The dNTPs, which are the building blocks used in amplifying nucleic acid molecules, can typically be supplied in standard PCR at a concentration of, for example, about 40-200 μM each of deoxyadenosine triphosphate (“dATP”), deoxyguanosine triphosphate (“dGTP”), deoxycytidine triphosphate (“dCTP”) and deoxythymidine triphosphate (“dTTP”). Other dNTPs, such as deoxyuridine triphosphate (“dUTP”), dNTP analogs (e.g., inosine), and conjugated dNTPs can also be used, and are encompassed by the term “dNTPs” as used herein. While use of dNTPs at concentrations of about 40-200 μM each can be amenable to the methods of this teaching, concentrations of dNTPs higher than about 200 μM each could be advantageous. Thus, in some embodiments of the methods of these teachings, the concentration of each dNTP is generally at least about 500 μM and can be up to about 2 mM. In some further embodiments, the concentration of each dNTP may range from about 0.5 mM to about 1 mM. Specific dNTP concentrations used for any multiplex amplification can vary depending on multiplex conditions, and can be determined empirically by one of skill in the art using standard laboratory procedures.

The enzyme that polymerizes the nucleotide triphosphates into the amplified products in PCR can be any DNA polymerase. The DNA polymerase can be, for example, any heat-resistant polymerase known in the art. Examples of some polymerases that can be used in this teaching are DNA polymerases from organisms such as Thermus aquaticus, Thermus thermophilus, Thermococcus litoralis, Bacillus stearothermophilus, Thermotoga maritima and Pyrococcus sp. The enzyme can be acquired by any of several possible methods; for example, isolated from the source bacteria, produced by recombinant DNA technology or purchased from commercial sources. Some examples of such commercially available DNA polymerases include AmpliTaq Gold® DNA polymerase; AmpliTaq® DNA Polymerase; AmpliTaq® DNA Polymerase Stoffel Fragment; rTth DNA Polymerase; and rTth DNA Polymerase, XL (all manufactured by Applied Biosystems, Foster City, Calif.) and Platinum Taq DNA polymerase (Invitrogen). Other examples of suitable polymerases include Tne, Bst DNA polymerase large fragment from Bacillus stearothermophilus, Vent and Vent Exo- from Thermococcus litoralis, Tma from Thermotoga maritima, Deep Vent and Deep Vent Exo- and Pfu from Pyrococcus sp., and mutants, variants and derivatives of the foregoing.

Other known components of PCR can be used within the scope of the present teachings. Some examples of such components include sorbitol, detergents (e.g., Triton X-100, Nonidet P-40 (NP-40), Tween-20) and agents that disrupt mismatching of nucleotide pairs, such as, for example, dimethylsulfoxide (DMSO), and tetramethylammonium chloride (TMAC), and uracil N-glycosylase or other agents which act to prevent amplicon contamination of the PCR and/or unwanted generation of product during incubation or preparation of the PCR, before the PCR procedure begins.

PCR cycle temperatures, the number of cycles and their durations can be varied to optimize a particular reaction, as a matter of routine experimentation. Those of ordinary skill in the art will recognize the following as guidance in determining the various parameters for PCR, and will also recognize that variation of one or more conditions is within the scope of the present teachings. Temperatures and cycle times are determined for three stages in PCR: denaturation, annealing and extension. One round of denaturation, annealing and extension is referred to as a “cycle.” Denaturation can generally be conducted at a temperature high enough to permit the strands of DNA to separate, yet not so high as to destroy polymerase activity. Generally, thermoresistant polymerases can be used in the reaction, which do not denature but retain some level of activity at elevated temperatures. However, heat-labile polymerases can be used if they are replenished after each denaturation step of the PCR. Typically, denaturation can be conducted above about 90° C. and below about 100° C. In some embodiments, denaturation can be conducted at a temperature of about 94-95° C. Denaturation of DNA can generally be conducted for at least about 1 to about 30 seconds. In some embodiments, denaturation can be conducted for about 1 to about 15 seconds. In other embodiments, denaturation can be conducted for up to about 1 minute or more. In addition to the denaturation of DNA, for some polymerases, such as AmpliTaq Gold® DNA polymerase, incubation at the denaturation temperature also can serve to activate the enzyme. Therefore, it can be advantageous to allow the first denaturation step of the PCR to be longer than subsequent denaturation steps when these polymerases are used.

During the annealing phase, oligonucleotide primers anneal to the target DNA in their regions of complementarity and are substantially extended by the DNA polymerase, once the latter has bound to the primer-template duplex. In a conventional PCR, the annealing temperature can typically be at or below the melting point (Tₘ) of the least stable primer-template duplex, where Tₘ can be estimated by any of several theoretical methods well known to practitioners of the art. For example, Tₘ can be determined by the formula:

Tₘ=(4° C.×number of G and C bases)+(2° C.×number of A and T bases).

Typically, in standard PCR, the annealing temperature can be about 5-10° C. below the estimated Tₘ of the least stable primer-template duplex. The annealing time can be between about 20-30 seconds and about 2 minutes. The annealing phase is typically followed by an extension phase. Extension can be conducted for a sufficient amount of time to allow the polymerase enzyme to complete primer extension into the appropriately sized amplification products.

The number of cycles in the PCR (one cycle comprising denaturation, annealing and extension) determines the extent of amplification and the subsequent amount of amplification product. PCR results in an exponential amplification of DNA molecules. Thus, theoretically, after each cycle of PCR there are twice the number of products that were present in the previous cycle, until PCR reagents are exhausted and a plateau is reached at which no further amplification products are generated. Typically, about 20-30 cycles of PCR may be performed to reach this plateau. More typically, about 25-30 cycles may be performed, although cycle number is not particularly limited.

For some embodiments, it can be advantageous to incubate the reactions at a certain temperature following the last phase of the last cycle of PCR. In some embodiments, a prolonged extension phase can be selected. In other embodiments, an incubation at a low temperature (e.g., about 4° C.) can be selected.

Various methods can be used to evaluate the products of the amplified alleles in the mixture of amplification products obtained from the multiplex reaction including, for example, detection of fluorescent labeled products, detection of radioisotope labeled products, silver staining of the amplification products, or the use of DNA intercalator dyes such as ethidium bromide (EtBr) and SYBR green cyanine dye to visualize double-stranded amplification products. Fluorescent labels suitable for attachment to primers for use in the present teachings are numerous, commercially available, and well-known in the art. With fluorescent analysis, at least one fluorescent labeled primer can be used for the amplification of each locus. Fluorescent detection may be desirable over radioactive methods of labeling and product detection, for example, because fluorescent detection does not require the use of radioactive materials, and thus avoids the regulatory and safety problems that accompany the use of radioactive materials. Fluorescent detection with labeled primers may also be selected over other non-radioactive methods of detection, such as silver staining and DNA intercalators, because fluorescent methods of detection generally reveal fewer amplification artifacts than do silver staining and DNA intercalators. This is due in part to the fact that only the amplified strands of DNA with labels attached thereto are detected in fluorescent detection, whereas both strands of every amplified product are stained and detected using the silver staining and intercalator methods of detection, which result in visualization of many non-specific amplification artifacts. Additionally, there are potential health risks associated with the use of EtBr and SYBR. EtBr is a known mutagen; SYBR, although less of a mutagen than EtBr, is generally suspended in DMSO, which can rapidly pass through skin.

Where fluorescent labeling of primers is used in a multiplex reaction, generally at least three different labels, at least four different labels, at least five different labels and at least six different labels can be used to label the different primers. When a size marker is used to evaluate the products of the multiplex reaction, the primers used to prepare the size marker may be labeled with a different label from the primers that amplify the loci of interest in the reaction. With the advent of automated fluorescent imaging and analysis, faster detection and analysis of multiplex amplification products can be achieved.

In some embodiments of the present teaching, a fluorophore can be used to label at least one primer of the multiplex amplification, e.g. by being covalently bound to the primer, thus creating a fluorescent labeled primer. In some embodiments, primers for different target loci in a multiplex can be labeled with different fluorophores, each fluorophore producing a different colored product depending on the emission wavelength of the fluorophore. These variously labeled primers can be used in the same multiplex reaction, and their respective amplification products subsequently analyzed together. Either the forward or reverse primer of the pair that amplifies a specific locus can be labeled, although the forward may more often be labeled.

The following are some examples of possible fluorophores well known in the art and suitable for use in the present teachings. The list is intended to be exemplary and is by no means exhaustive. Some possible fluorophores include: fluorescein (FL), which absorbs maximally at 492 nm and emits maximally at 520 nm; N,N,N′,N′-tetramethyl-6-carboxyrhodamine (TAMRA™), which absorbs maximally at 555 nm and emits maximally at 580 nm; 5-carboxyfluorescein (5-FAM™), which absorbs maximally at 495 nm and emits maximally at 525 nm; 2′,7′-dimethoxy-4′,5′-dichloro-6-carboxyfluorescein (JOE™), which absorbs maximally at 525 nm and emits maximally at 555 nm); 6-carboxy-X-rhodamine (ROX™), which absorbs maximally at 585 nm and emits maximally at 605 nm; CY3™, which absorbs maximally at 552 nm and emits maximally at 570 nm; CY5™, which absorbs maximally at 643 nm and emits maximally at 667 nm; tetrachloro-fluorescein (TET™), which absorbs maximally at 521 nm and emits maximally at 536 nm; and hexachloro-fluorescein (HEX™), which absorbs maximally at 535 nm and emits maximally at 556 nm; NED™, which absorbs maximally at 546 nm and emits maximally at 575 nm; 6-FAM™, which emits maximally at approximately 520 nm; VIC® which emits maximally at approximately 550 nm; PET® which emits maximally at approximately 590 nm; and LIZ™, which emits maximally at approximately 650 nm. See S R Coticone et al., U.S. Pat. No. 6,780,588; AMPFLSTR® IDENTIFILER™ PCR AMPLIFICATION KIT USER'S MANUAL, pp. 1-3, Applied Biosystems (2001). Note that the above listed emission and/or absorption wavelengths are typical and can be used for general guidance purposes only; actual peak wavelengths may vary for different applications and under different conditions. Additional fluorophores can be selected for the desired absorbance and emission spectra as well as color as is known to one of skill in the art and are provided below:

TABLE 2
Commercially Available Dyes

| | Abs | Em | | Abs | Em |
|---|---|---|---|---|---|
| Fluorophore | (nm) | (nm) | Fluorophore | (nm) | (nm) |
| Methoxycoumarin | 340 | 405 | Dansyl | 340 | 520 |
| Pyrene | 345 | 378 | Alexa Fluor ® 350 | 346 | 442 |
| CF ™ 350 | 347 | 448 | AMCA | 349 | 448 |
| DyLight 350 | 353 | 432 | Marina Blue ® dye | 365 | 460 |
| Dapoxyl ® | 373 | 551 | Dialkylamino- | 375 | 470-475 |
| dye | | | coumarin | 435 | |
| Bimane | 380 | 458 | SeTau 380 | 381 | 480 |
| Hydroxycoumarin | 385 | 445 | ATTO 390 | 390 | 479 |
| Cascade Blue ® | 400 | 420 | Pacific Orange ® | 400 | 551 |
| dye | | | dye | | |
| DyLight ® 405 | 400 | 420 | Alexa Fluor ® 405 | 402 | 421 |
| SeTau 404 | 402 | 518 | Cascade Yellow ® | 402 | 545 |
| | | | dye | | |
| CF ™ 405S | 404 | 431 | CF ™ 405M | 408 | 452 |
| Pacific Blue ™ | 410 | 455 | PyMPO | 415 | 570 |
| dye | | | | | |
| DY-415 | 415 | 467 | SeTau 425 | 425 | 545 |
| Alexa Fluor ® | 434 | 539 | ATTO 425 | 436 | 484 |
| 430 | | | | | |
| ATTO 465 | 453 | 508 | NBD | 465 | 535 |
| Seta 470 | 469 | 521 | CF ™ 485 | 470-488 | 513 |
| DY-485XL | 485 | 560 | CF ™ 488A | 490 | 515 |
| DyLight ® 488 | 493 | 518 | DY 496 | 493 | 521 |
| Fluorescein | 494 | 518 | ATTO 495 | 495 | 527 |
| Alexa Fluor ® | 495 | 519 | Oregon Green ® | 496 | 524 |
| 488 | | | 488 | | |
| BODIPY ® | 500 | 506 | CAL Fluor ® Green | 500 | 522 |
| 493/503 | | | 520 | | |
| DY-480XL | 500 | 630 | ATTO 488 | 501 | 523 |
| Rhodamine Green | 502 | 527 | BODIPY ® FL | 505 | 513 |
| dye | | | | | |
| DY 505 | 505 | 530 | DY 510XL | 509 | 590 |
| 2′,7′- | 510 | 532 | Oregon Green ® | 511 | 530 |
| Dichloro- | | | 514 | | |
| fluorescein | | | | | |
| DY-481XL | 515 | 650 | ATTO 520 | 516 | 538 |
| Alexa Fluor ® | 518 | 540 | CAL Fluor ® Gold | 519 | 537 |
| 514 | | | 540 | | |
| DY 520XL | 520 | 664 | 4′,5′-Dichloro- | 522 | 550 |
| | | | 2′,7′-dimethoxy- | | |
| | | | fluorescein (JOE) | | |
| DY-521XL | 523 | 668 | Eosin | 524 | 544 |
| Rhodamine 6G | 525 | 555 | BODIPY ® R6G | 528 | 550 |
| Alexa Fluor ® | 531 | 554 | ATTO 532 | 532 | 553 |
| 532 | | | | | |
| BODIPY ® | 534 | 554 | CAL Fluor ® | 534 | 556 |
| 530/550 | | | Orange 560 | | |
| DY-530 | 539 | 561 | BODIPY ® TMR | 542 | 574 |
| DY-555 | 547 | 572 | DY556 | 548 | 573 |
| Quasar ® 570 | 548 | 570 | Cy 3 | 550 | 570 |
| CF ™555 | 550 | 570 | DY-554 | 551 | 572 |
| DY 550 | 553 | 578 | ATTO 550 | 554 | 576 |
| Tetramethyl- | 555 | 580 | Alexa Fluor ® 555 | 555 | 565 |
| rhodamine (TMR) | | | | | |
| Seta 555 | 556 | 570 | Alexa Fluor ® 546 | 556 | 575 |
| DY-547 | 557 | 574 | DY-548 | 558 | 572 |
| BODIPY ® | 558 | 569 | DY-560 | 559 | 578 |
| 558/568 | | | | | |
| DY 549 | 560 | 575 | DyLight ® 549 | 562 | 618 |
| CF ™ 568 | 562 | 583 | ATTO 565 | 563 | 592 |
| BODIPY ® | 565 | 571 | CAL Fluor ® Red | 566 | 588 |
| 564/570 | | | 590 | | |
| Lissamine | 570 | 590 | Rhodamine Red | 570 | 590 |
| rhodamine B | | | dye | | |
| BODIPY ® | 576 | 590 | Alexa Fluor ® 568 | 578 | 603 |
| 576/589 | | | | | |
| X-rhodamine | 580 | 605 | DY-590 | 580 | 599 |
| BODIPY ® | 584 | 592 | CAL Fluor ® Red | 587 | 608 |
| 581/591 | | | 610 | | |
| BODIPY ® TR | 589 | 617 | Alexa Fluor ® 594 | 590 | 617 |
| ATTO 590 | 594 | 624 | CF ™ 594 | 594 | 614 |
| CAL Fluor ® | 595 | 615 | Texas Red ® dye | 595 | 615 |
| Red 615 | | | | | |
| Naphtho- | 605 | 675 | DY-682 | 609 | 709 |
| fluorescein | | | | | |
| DY-610 | 610 | 630 | CAL Fluor ® Red | 611 | 631 |
| | | | 635 | | |
| ATTO 611x | 611 | 681 | Alexa Fluor ® 610 | 612 | 628 |
| ATTO 610 | 615 | 634 | CF ™ 620R | 617 | 639 |
| ATTO 620 | 619 | 643 | DY-615 | 621 | 641 |
| BODIPY ® | 625 | 640 | ATTO 633 | 629 | 657 |
| 630/650 | | | | | |
| CF ™ 633 | 630 | 650 | Seta 632 | 632 | 641 |
| Alexa Fluor ® | 632 | 647 | Alexa Fluor ® 635 | 633 | 647 |
| 633 | | | | | |
| DY-634 | 635 | 658 | Seta 633 | 637 | 647 |
| DY-630 | 636 | 657 | DY-633 | 637 | 657 |
| DY-632 | 637 | 657 | DyLight ® 633 | 638 | 658 |
| Seta 640 | 640 | 656 | CF ™ 640R | 642 | 662 |
| ATTO 647N | 644 | 669 | Quasar ® 670 | 644 | 670 |
| ATTO 647 | 645 | 669 | DY-636 | 645 | 671 |
| BODIPY ® | 646 | 660 | Seta 646 | 646 | 656 |
| 650/665 | | | | | |
| DY-635 | 647 | 671 | Square 635 | 647 | 666 |
| Cy 5 | 649 | 650/ | Alexa Fluor ® 647 | 650 | 668 |
| | | 670 | | | |
| CF ™ 647 | 650 | 665 | Seta 650 | 651 | 671 |
| Square 650 | 653 | 671 | DY-647 | 653 | 672 |
| DY-648 | 653 | 674 | DY-650 | 653 | 674 |
| DyLight ® 649 | 654 | 673 | DY-652 | 654 | 675 |
| DY-649 | 655 | 676 | DY-651 | 656 | 678 |
| Square 660 | 658 | 677 | Seta 660 | 661 | 672 |
| Alexa Fluor ® | 663 | 690 | ATTO 655 | 663 | 684 |
| 660 | | | | | |
| Seta 665 | 667 | 683 | Square 670 | 667 | 685 |
| Seta 670 | 667 | 686 | DY-675 | 674 | 699 |
| DY-677 | 673 | 694 | DY-676 | 674 | 699 |
| Alexa Fluor ® | 679 | 702 | IRDye ® 700DX | 680 | 687 |
| 680 | | | | | |
| ATTO 680 | 680 | 700 | CF ™ 680R | 680 | 701 |
| CF ™ 680 | 681 | 698 | Square 685 | 683 | 703 |
| DY-680 | 690 | 709 | DY-681 | 691 | 708 |
| DyLight ® | 692 | 712 | Seta 690 | 693 | 714 |
| 680 | | | | | |
| ATTO 700 | 700 | 719 | Alexa Fluor ® 700 | 702 | 723 |
| Seta 700 | 702 | 728 | ATTO 725 | 725 | 752 |
| ATTO 740 | 740 | 764 | Alexa Fluor ® 750 | 749 | 775 |
| Seta 750 | 750 | 779 | DyLight ® 750 | 752 | 778 |
| CF ™ 750 | 755 | 777 | CF ™ 770 | 770 | 797 |
| DyLight ® 800 | 777 | 794 | IRDye ®800RS | 770 | 786 |
| IRDye ® | 778 | 794 | Alexa Fluor ® 790 | 782 | 805 |
| 800 CW | | | | | |
| CF ™ 790 | 784 | 806 | | | |

Various embodiments of the present teachings may comprise a single multiplex reaction comprising at least five different dyes. Dyes TMR-ET, CXR-ET and CC5 are also used (Promega, Madison, Wis.). The at least four dyes may comprise any four of the above-listed dyes, or any other four dyes known in the art, or 6-FAM™, VIC®, NED™ and PET®. Other embodiments of the present teaching may comprise a single multiplex reaction comprising at least five different dyes. These at least five dyes may comprise any five of the above-listed dyes, or any other five dyes known in the art, or 6-FAM™, VIC®, NED™, PET®, and LIZ™ dyes. Other embodiments of the present teaching may comprise a single multiplex reaction comprising at least six different dyes. These at least six dyes may comprise any six of the above-listed dyes, or any other six dyes known in the art, 6-FAM™, VIC®, NED™, PET®, SID™ and LIZ™ dyes with the SID dye having a maximum emission at approximately 620 nm (LIZ™ dye was used to label the size standards). TAZ™ dye can also be used (Applied Biosystems).

The PCR products can be analyzed on a sieving or non-sieving medium. In some embodiments of these teachings, for example, the PCR products can be analyzed by electrophoresis; e.g., capillary electrophoresis, as described in H. Wenz et al. (1998), GENOME RES. 8:69-80 (see also E. Buel et al. (1998), J. FORENSIC SCI. 43:(1), pp. 164-170)), or slab gel electrophoresis, as described in M. Christensen et al. (1999), SCAND. J. CLIN. LAB. INVEST. 59(3): 167-177, or denaturing polyacrylamide gel electrophoresis (see, e.g., J. Sambrook et al. (1989), in MOLECULAR CLONING: A LABORATORY MANUAL, SECOND EDITION, Cold Spring Harbor Laboratory Press, Cold Spring Harbor, N.Y., pp. 13.45-13.57). The separation of DNA fragments in electrophoresis is based primarily on differential fragment size. Amplification products can also be analyzed by chromatography; e.g., by size exclusion chromatography (SEC). In some embodiments, the PCR products can be analyzed by a mobility-dependent separation method such as electrophoresis and chromatography as described above.

Once the amplified alleles are separated, these alleles and any other DNA in, for example, the gel or capillary (e.g., a DNA size markers or an allelic ladder) can then be visualized and analyzed. Visualization of the DNA can be accomplished using any of a number of techniques known in the art, such as, for example, silver staining or by use of reporters such as radioisotopes and fluorescent dyes, as described herein, or chemiluminescers and enzymes in combination with detectable substrates. Oftentimes, the method for detection of multiplex loci can be by fluorescence. See, e.g., J W Schumm et al. in PROCEEDINGS FROM THE EIGHTH INTERNATIONAL SYMPOSIUM ON HUMAN IDENTIFICATION, pub. 1998 by Promega Corporation, pp. 78-84; E. Buel et al. (1998), supra. Where fluorescent-labeled primers are used for detecting each locus in the multiplex reaction, amplification can be followed by detection of the labeled products employing a fluorometric detector. See the description of fluorescent dyes, supra.

The size of the alleles present at each locus in the DNA sample can be determined by comparison to a size standard in electrophoresis, such as a DNA marker of known size. Markers for evaluation of a multiplex amplification containing two or more polymorphic STR loci may also comprise a locus-specific allelic ladder or a combination of allelic ladders for each of the loci being evaluated. See, e.g., C. Puers et al. (1993), AM. J. HUM. GENET. 53:953-958; C. Puers et al. (1994), GENOMICS 23:260-264. See also, U.S. Pat. Nos. 5,599,666; 5,674,686; and 5,783,406 for descriptions of some allelic ladders suitable for use in the detection of STR loci, and some methods of ladder construction disclosed therein. Following the construction of allelic ladders for individual loci, the ladders can be electrophoresed at the same time as the amplification products. Each allelic ladder co-migrates with the alleles from the corresponding locus.

The products of the multiplex reactions of the present teachings can also be evaluated using an internal lane standard; i.e., a specialized type of size marker configured to be electrophoresed, for example, in the same capillary as the amplification products. The internal lane standard can comprise a series of fragments of known length. The internal lane standard can also be labeled with a fluorescent dye, which is distinguishable from other dyes in the amplification reaction. The lane standard can be mixed with amplified sample or size standards/allelic ladders and electrophoresed with either, in order to compare migration in different lanes of gel electrophoresis or different capillaries of capillary electrophoresis. Variation in the migration of the internal lane standard can serve to indicate variation in the performance of the separation medium. Quantitation of this difference and correlation with the allelic ladders can provide for calibration of amplification product electrophoresed in different lanes or capillaries, and correction in the size determination of alleles in unknown samples.

Where fluorescent dyes are used to label amplification products, the electrophoresed and separated products can be analyzed using fluorescence detection equipment such as, for example, the ABI PRISM® 310 3130xl, or 3500XL Genetic Analyzers, or an ABI PRISM® 377 DNA Sequencer (Applied Biosystems, Foster City, Calif.); or a Hitachi FMBIO™ II Fluorescent Scanner (Hitachi Software Engineering America, Ltd., South San Francisco, Calif.). In various embodiments of the present teachings, PCR products can be analyzed by a capillary gel electrophoresis protocol in conjunction with such electrophoresis instrumentation as the ABI PRISM®3130xl and 3500XLGenetic Analyzer (Applied Biosystems), and allelic analysis of the electrophoresed amplification products can be performed, for example, with GeneMapper® ID-X Software v1.2, from Applied Biosystems. In other embodiments, the amplification products can be separated by electrophoresis in, for example, about a 4.5%, 29:1 acrylamide:bis acrylamide, 8 M urea gel as prepared for an ABI PRISM® 377 Automated Fluorescence DNA Sequencer.

The present teachings are also directed to kits that utilize the processes described above. In some embodiments, a basic kit can comprise a container having one or more locus-specific primers. A kit can also optionally comprise instructions for use. A kit can also comprise other optional kit components, such as, for example, one or more of an allelic ladder directed to each of the specified loci, a sufficient quantity of enzyme for amplification, amplification buffer to facilitate the amplification, divalent cation solution to facilitate enzyme activity, dNTPs for strand extension during amplification, loading solution for preparation of the amplified material for electrophoresis, genomic DNA as a template control, a size marker to insure that materials migrate as anticipated in the separation medium, and a protocol and manual to educate the user and limit error in use. The amounts of the various reagents in the kits also can be varied depending upon a number of factors, such as the optimum sensitivity of the process. It is within the scope of these teachings to provide test kits for use in manual applications or test kits for use with automated detectors or analyzers.

Personal identification tests, or DNA typing, can be performed on any specimen that contains nucleic acid, such as bone, hair, blood, tissue and the like. DNA can be extracted from the specimen and a panel of primers used to amplify a desired set of STR loci of the DNA in a multiplex to generate a set of amplification products, as described herein. In forensic testing, the particular specimen's amplification pattern, or DNA profile, can be compared with a known sample taken from the presumptive victim (the presumed matching source), or can be compared to the pattern of amplified loci derived from the presumptive victim's family members (e.g., the mother and/or father) wherein the same set of STR loci is amplified. The pattern of STR loci amplification can be used to confirm or rule out the identity of the victim. In paternity testing, the test specimen generally can be from the child and comparison can be made to the STR loci pattern from the presumptive father, and/or can be matched with the STR loci pattern from the child's mother. The pattern of STR loci amplification can be used to confirm or rule out the identity of the father. The amplification and comparison of specific loci can also be used in paternity testing in a breeding context; e.g., for cattle, dogs, horses and other animals. C R Primmer et al. (1995), MOL. ECOL. 4:493-498.

In a clinical setting, such STR markers can be used, for example, to monitor the degree of donor engraftment in bone marrow transplants. In hospitals, these markers can also be useful in specimen matching and tracking. These markers have also entered other fields of science, such as population biology studies on human racial and ethnic group differences (D B Goldstein et al. (1995), PROC. NATL. ACAD. SCI. U.S.A. 92:6723-6727), evolution and species divergence, and variation in animal and plant taxa (M W Bruford et al. (1993), CURR. BIOL. 3:939-943).

The reference works, patents, patent applications, scientific literature and other printed publications, as well as accession numbers to GenBank database sequences that are referred to herein, are all hereby incorporated by reference in their entirety.

### EXAMPLES

Aspects of the present teachings can be further understood in light of the following examples, which should not be construed as limiting the scope of the present teachings in any way.

### Example I

In certain embodiments, a DNA sample to be analyzed was combined with STR- and Amelogenin-specific primer sets in a PCR mixture to amplify the Identifiler® loci D7S820, D5S818, D13S317, D16S539, D18S51, D19S433, D21S11, D2S1338, D3S1358, D8S1179, CSF1PO, FGA, TH01, TPOX, VWA, Amelogenin, and five new STR loci D10S1248, D12S391, D1S1656, D22S1045, and D2S441. Primer sets for these loci were designed according to the methodology provided herein, supra. One primer from each of the primer sets that amplify D3S1358, VWA, TPOX, and D7S820 was labeled with the 6-FAM™ fluorescent label. One primer from each of the primer sets that amplify Amelogenin, D5S818, D21S11, and D18S51 was labeled with the VIC® fluorescent label. One primer from each of the primer sets that amplify D2S441, D19S433, TH01 and FGA was labeled with the TED™ fluorescent label. One primer from each of the primer sets that amplify D22S1045, D8S1179, D13S317, D16S539, and D2S1388 was labeled with the TAZ® fluorescent label. One primer from each of the primer sets that amplify D10S1248, D1S1656, D12S391, and CSF1PO was labeled with the SID®fluorescent label. A sixth fluorescent label, LIZ™ dye, was used to label a size standard.

### PCR Assay Set-Up

Methods of the disclosed present teachings can be practiced as taught in the AmpFISTR® NGM SElect™ PCR Amplification Kit User's Guide, PN 4425511 (Applied Biosystems), incorporated herein by reference. The recommended PCR conditions call for 1.0 ng of human genomic DNA to be amplified in a total reaction volume of 25 μL. A PCR reaction mix is prepared based on the following calculation per reaction:

| Component | Volume per Reaction |
|---|---|
| NGM Master Mix (2.5X) | 10 μl |
| Above Primer Set (5X) | 5 μl |

An additional 3 reactions are included in the calculation to provide excess volume for the loss that occurs during reagent transfers. Again, thorough mixing by vortexing at medium speed for 10 sec. followed by briefly centrifuging to remove any liquid from the cap of the vial containing the PCR reaction mix. 15 μL of the PCR reaction mix is aliquoted into each reaction vial or well followed by addition of each sample to be analyzed into its own vial or well, up to 10 μL volume to have approximately 1.0 ng sample DNA/reaction. Samples of less than 10 μL are made up to a final 10 μL volume with Low-TE Buffer (consisting of 10 mM Tris-CI pH 8.0 and 0.1 mM EDTA, was added as needed to bring the reaction volume up to 25 μL). Following sample addition the tubes or wells are covered and a brief centrifugation at 3000 rpm for about 30 seconds is performed to remove any air bubbles prior to amplification.

A 25-marker multiplex was prepared using the NGM kit PCR master mix and PCR cycling conditions. Primer concentrations were adjusted in the master mix and were at a final concentration of from 0.05 uM to 0.30 uM in a 25 ul reaction volume to achieve optimum color balance, sensitivity and peak heights within detectable limits. Capillary electrophoresis was performed on the 3500XL instrument (Applied Biosystems) with an injection at 1.2 kV for 24 seconds. Results are shown in FIG. 2.

### PCR Reaction Parameters

PCR reactions were set up in MicroAmp® 96-well reaction plates covered by either MicroAmp® 8-cap strips or MicroAmp® Clear Adhesive Film. The samples are amplified according to specifications found in the User Guide above. When using the GeneAmp PCR System 9700 with either 96-well silver or gold-plated silver block, select the 9600 Emulation Mode. Thermal cycling conditions are an initial incubation step at 95° C. for 11 min., 28 cycles of 94° C. for 20 sec. denaturing and 59° C. for 3 min. annealing (2 min. for a 25-multiplex) followed by a final extension at 60° C. for 10 min. and final hold at 4° C. indefinitely. Following completion, the samples should be protected from light and stored at 2 to 8° C. if the amplified DNA will be analyzed within 2 weeks or at −15 to −20° C. if use is greater than 2 weeks.

### Capillary Electrophoresis Sample Preparation and Detection

The amplified samples are analyzed by methods that resolve amplification product size and/or sequence differences as would be known to one of skill in the art. For example, capillary electrophoresis can be used following the instrument manufactures directions. Briefly, 0.5 μL GeneScan™-600 LIZ™ Size Standard and 8.5 μL of Hi-Di™ Formamide are mixed for each sample to be analyzed. 9.0 μL of the Formamide/GeneScan-600 LIZ solution is dispensed into each well of a MicroAmp® Optical 96-well reaction plate to which a 1.0 μL aliquot of the PCR amplified sample or allelic ladder is added and the plate is covered. The plate is briefly centrifuged to mix the contents and collect them at the bottom of the plate. The plate is heated at 95° C. for 3 minutes to heat-denature the samples and then quenched immediately by placing on ice for 3 minutes.

### Capillary Electrophoresis Methods and Analysis

Capillary electrophoresis (CE) was performed on the current Applied Biosystems instruments: the Applied Biosystems 3500xl Genetic Analyzer using the specified J6 variable binning module as described in the instrument's User's Guide. The 3500xl Genetic Analyzer's parameters were: sample injection for 24 sec at 1.2 kV and electrophoresis at 15 kV for 1210 sec in Performance Optimized Polymer (POP-4™ polymer) with a run temperature of 60° C. as indicated in the HID36_POP4xl_G5_NT3200 protocol. Variations in instrument parameters, e.g. injection conditions, were different on other CE instruments such as the 3500, 3130xl, or 3130 Genetic Analyzers.) The data were collected using versions the Applied Biosystems Data Collection Software specific to the different instruments, such as v.3.0 for the 3130xl and 3500 Data Collection Software v1.0 that were analyzed using GeneMapper ID-X v1.2. FIG. 1 provides the spacing of an exemplary 21-plex multiplex of the present teachings.

Following instrument set-up according to the manufacturer's directions each sample is injected and analyzed by appropriate software, e.g., GeneMapper® ID Software v3.2 or GeneMapper® ID-X v1.2 software with the standard analysis settings. A peak amplitude of 50 RFU (relative fluorescence units) was used as the peak detection threshold.

### Example II

In certain embodiments, a DNA sample to be analyzed was combined with STR-, a Y indel- and Amelogenin-specific primer sets in a PCR mixture to amplify the Identifiler® loci D7S820, D5S818, D13S317, D16S539, D18S51, D19S433, D21S11, D2S1338, D3S1358, D8S1179, CSF1PO, FGA, TH01, TPOX, VWA, Amelogenin, and seven new STR loci D10S1248, D12S391, D1S1656, D22S1045, D2S441 and Penta E along with Y STR DYS391. Primer sets for these loci were designed according to the methodology provided herein, supra. One primer from each of the primer sets that amplify D3S1358, VWA, TPOX, D7S820, and DYS391 was labeled with the 6-FAM™ fluorescent label. One primer from each of the primer sets that amplify Amelogenin, D5S818, D21S11, and D18S51 was labeled with the VIC® fluorescent label. One primer from each of the primer sets that amplify D2S441, D19S433, TH01 and FGA was labeled with the TED™ fluorescent label. One primer from each of the primer sets that amplify D22S1045, D8S1179, D13S317, D16S539 and D2S1338 was labeled with the TAZ® fluorescent label. One primer from each of the primer sets that amplify D10S1248, D1S1656, D12S391, CSF and Penta E was labeled with the SID® fluorescent label. A sixth fluorescent label, LIZ™ dye, was used to label a size standard. PCR as described above for casework samples in which the DNA was extracted or as described below for database samples in which direct amplification of the sample was performed (the sample is not extracted from the substrate upon which it was either collected or swabbed onto in the case of paper or the swab itself) as described below.

### PCR Reaction Parameters for Direct Amplification

PCR reactions were set up in MicroAmp® 96-well reaction plates covered by either MicroAmp® 8-cap strips or MicroAmp® Clear Adhesive Film. The samples are amplified according to the following specifications: Amplification was performed on a Veriti® 96-well Thermal Cycler (PN 4375786, Applied Biosystems). Thermal cycling conditions are an initial incubation step at 95° C. for 1 min., 26 cycles of 94° C. for 3 sec. denaturing at 60° C. for 30 sec. followed by a final extension at 60° C. for 5 min. and final hold at 4° C. indefinitely. Following completion, the samples should be protected from light and stored at 2 to 8° C. if the amplified DNA will be analyzed within 2 weeks or at −15 to −20° C. if use is greater than 2 weeks. Thermal cycling cycle determination should be determined for each laboratory according to their internal validation criteria and can be from 25 to 28 cycles with a total cycling time of about 30 to 38 min.

### Capillary Electrophoresis Sample Preparation and Detection

The amplified samples are analyzed by methods that resolve amplification product size and/or sequence differences as would be known to one of skill in the art. The following directions were used on the Applied Biosystems 3500 and 3500xL Genetic Analyzers. Additional information on setting up the instrument can be found in the User Guide (PN 4401661 Applied BioSystems). For example, capillary electrophoresis can be used following the instrument manufactures directions. Briefly, 0.5 μL GeneScan™-600 LIZ™ Size Standard and 9.5 μL of Hi-Di™ Formamide are mixed for each sample to be analyzed. 10.0 μL of the Formamide/GeneScan-600 LIZ solution is dispensed into each well of a MicroAmp® Optical 96-well reaction plate to which a 1.0 μL aliquot of the PCR amplified sample or allelic ladder is added and the plate is covered. The plate is briefly centrifuged to mix the contents and collect them at the bottom of the plate. The plate is heated at 95° C. for 3 minutes to heat-denature the samples and then quenched immediately by placing on ice for 3 minutes.

### Capillary Electrophoresis Methods and Analysis

Capillary electrophoresis (CE) was performed on the current Applied Biosystems instruments: the Applied Biosystems 3500xL Genetic Analyzer using the specified J6 variable binning module as described in the instrument's User's Guide. The 3500xl Genetic Analyzer's parameters were: sample injection for 24 sec at 1.2 kV and electrophoresis at 15 kV for 1210 sec in Performance Optimized Polymer (POP-4™ polymer) with a run temperature of 60° C. as indicated in the HID36_POP4xl_G5_NT3200 protocol. Variations in instrument parameters, e.g. injection conditions, were different on other CE instruments such as the 3500, 3130xl, or 3130 Genetic Analyzers.) The data were collected using versions the Applied Biosystems Data Collection Software specific to the different instruments, such as v.3.0 for the 3130xl and 3500 Data Collection Software v1.0 that were analyzed using GeneMapper ID-X v1.2. FIG. 1 provides the spacing of an exemplary 21-plex multiplex of the present teachings.

Following instrument set-up according to the manufacturer's directions each sample was injected and analyzed by appropriate software, e.g., GeneMapper® ID Software v3.2 or GeneMapper® ID-X v1.2 software with the standard analysis settings. A peak amplitude of 50 RFU (relative fluorescence units) was used as the peak detection threshold.

### Example III

In certain embodiments, a DNA sample to be analyzed was combined with STR-, a Y indel- and Amelogenin-specific primer sets in a PCR mixture to amplify the Identifiler® loci D7S820, D5S818, D13S317, D16S539, D18S51, D19S433, D21S11, D2S1338, D3S1358, D8S1179, CSF1PO, FGA, TH01, TPOX, VWA, Amelogenin, and seven new STR loci D10S1248, D12S391, D1S1656, D22S1045, D2S441, DYS391 and SE33 along with Y indel rs 2032678. A direct substitution of the STR marker D6S1043 can be made for SE33. D6S1043 is highly polymorphic among persons of Asian decent. Primer sets for these loci were designed according to the methodology provided herein, supra. One primer from each of the primer sets that amplify D3S1358, VWA, D16S539, CSF1PO and TPOX was labeled with the 6-FAM™ fluorescent label. One primer from each of the primer sets that amplify Y indel rs 2032678, Amelogenin, D8S1179, D21S11, D18S51 and DYS391 was labeled with the VIC® fluorescent label. One primer from each of the primer sets that amplify D2S441, D19S433, TH01 and FGA was labeled with the TED™ fluorescent label. One primer from each of the primer sets that amplify D22S1045, D5S818, D13S317, D7S820 and SE33 was labeled with the TAZ® fluorescent label. One primer from each of the primer sets that amplify D10S1248, D1S1656, D12S391, and D2S1338 was labeled with the SID® fluorescent label. A sixth fluorescent label, LIZ™ dye, was used to label a size standard.

### Example IV

The following procedures are representative of procedures that can be employed for collection of nucleic acid from a biological sample for processing by direct amplification.

### DNA Samples

Anonymous whole-blood samples were purchased from Seracare Life Sciences (Oceanside, Calif.) or Interstate Blood Bank, Inc. (Memphis, Tenn.), and the control DNA 9947A was purchased from Marligen Biosciences (Ijamsville, Md.). FTA_cards, Indicating FTA cards, and EasiCollect devices were purchased from Whatman, Inc. Blood on FTA cards was prepared by spotting 75-80 uL of whole blood onto the center of the sampling spot. Buccal cells were collected using Buccal DNA Collector® (Bode Technology) EasiCollect devices or foam swabs, followed by contact transfer to the Indicating FTA_cards. PCR reaction conditions and capillary electrophoresis conditions as described in Example III.

Sample Processing from FTA Card for Direct Amplification, Database Samples

Buccal or Blood were spotted on FTA paper samples. A 1.2 mm punch was removed from the center of the sample and placed into individual wells of a MicroAmp® Optical 96-well reaction plate. Manual punching was performed by placing the tip of a 1.2 mm Harris Micro-Punch on the card holding the barrel of the Harris Micro-Punch (do not touch the plunger) and gently pressing and twisting ¼-turn to cut the 1.2 mm punch which was then ejected into the appropriate well on the reaction plate. If automated punching is used refer to the User Guide of your automated or semi-automated disc punch instrument (e.g. BSD 600) for proper guidance. It is appropriate to make the punch as close as possible to the center of the sample to ensure optimum peak intensity. It is noted that increasing the size of the punch may cause inhibition during the PCR amplification phase of the assay and reduce the quality and reproducibility of the result. 10 uL of NGM®SElect™ Express 2.5× Direct PCR master mix for STR analysis with Platinum Taq (NGM kit from Applied Biosystems, Platinum Taq available from Invitrogen, Carlsbad, Calif.) and 10 uL 2.5× Primer Mix (P/N 4472197, Applied Biosystems) was added to each well. The final volume was adjusted to 25 uL with low TE buffer or sterile water. PCR reaction conditions and capillary electrophoresis conditions were as described in Example III.

Sample Processing from Non-FTA Paper for Direct Amplification, Database Samples

Buccal or Blood were spotted on non-FTA paper samples. A 1.2 mm punch was removed from the center of the sample and placed into individual wells of a MicroAmp® Optical 96-well reaction plate as described for samples spotted onto FTA paper. 2 uL Prep-n-Go buffer was added to each well containing the 1.2 mm disc. 10 uL of NGM®SElect™ Express 2.5× Direct PCR master mix for STR analysis with Platinum Taq (NGM kit from Applied Biosystems, Platinum Taq available from Invitrogen, Carlsbad, Calif.) and 10 uL 2.5× Primer Mix (P/N 4472197, Applied Biosystems) was added to each well. The final volume was adjusted to 25 uL with low TE buffer or sterile water. PCR reaction conditions and capillary electrophoresis conditions were as described in Example III.

Sample Extraction from Swab for Direct Amplification, Database Samples

The swab head (either full or half) was placed into 400 uL of Prep-n-Go™ buffer (PN 4467082, Applied Biosystems) within a 96 deep well plate and incubated at room temperature for 20 minutes (an alternative throughput workflow would be to swirl for 10 seconds). 2-5 uL of cell lysate was added to a 96-well PCR plate containing 10 uL of NGM®SElect™ Express 2.5× Direct PCR master mix for STR analysis with Platinum Taq (NGM kit from Applied Biosystems, Platinum Taq available from Invitrogen, Carlsbad, Calif.) and 10 uL 2.5× Primer Mix (P/N 4472197, Applied Biosystems). PCR reaction conditions and capillary electrophoresis conditions were as described in Example III.

As those skilled in the art will appreciate, numerous changes and modifications may be made to the various embodiments of the present teachings without departing from the spirit of these teachings. It is intended that all such variations fall within the scope of these teachings.

We claim:

1. A composition for genotyping nucleic acid from a sample comprising:
a. amplifying the nucleic acid with a plurality of amplification primer pairs to form a plurality of amplification products; wherein at least one of each of said primer pairs comprises one of at least five different labels; wherein each of said amplification products comprise a different STR marker yielding an STR marker amplification product;
b. separating each of the STR marker amplification products by a mobility-dependent separation method; wherein:
i. a first primer set labeled with a first label comprises at least three different STR marker amplification products selected from D3S1358, vWA, TPOX, D16S539, CSF1PO, DYS391, and D7S820;
ii. a second primer set labeled with a second label comprises at least three different STR marker amplification products selected from D5S818, D21 S11, D8S1179, and D18S51, Y indel rs 2032678 and a sex-determination marker AMEL;
iii. a third primer set labeled with a third label comprises at least three different STR marker amplification products selected from D2S441, D19S433, TH01 and FGA;
iv. a fourth primer set labeled with a fourth label comprises at least three different STR marker amplification products D22S1045, D5S818, D8S1179, D13S317, D16S539, D2S1338, D7S820, D6S1043, and SE33; and
v. a fifth primer set labeled with a fifth label comprises at least three different STR marker amplification products selected from D10S1248, D1S1656, D12S391, CSF1PO, D2S1338, and Penta E; and
c. determining the genotype of the nucleic acid from the sample by identifying each allele(s) for each of said different STR marker amplification products.

2. The composition of claim 1, wherein at least four primer sets comprise at least four different STR marker amplification products.

3. The composition of claim 1, wherein D5S818 in the second primer set can also be so labeled such that it can be substituted for D8S1179 in the fourth primer set and D8S1179 can be so labeled such that it can be substituted for D5S818 in the second primer set.

4. The composition of claim 1, wherein D7S820 in the first primer set can also be so labeled such that it can be substituted for D21S11 in the second primer set or CSF1PO in the fifth primer set and D21S11 can be so labeled such that it can be substituted for D7S820 in the first primer set or CSF1PO in the fifth primer set and CSF1PO can be so labeled such that it can be substituted for D7S820 in the first primer set or D21S11 in the second label channel.

5. The composition of claim 1, wherein the nucleic acid is DNA, cDNA or RNA.

6. The composition of claim 1, wherein the sample is selected from whole blood, a tissue biopsy, lymph, bone, bone marrow, tooth, amniotic fluid, hair, skin, semen, anal secretions, vaginal secretions, perspiration, saliva, buccal swabs, various environmental samples (for example, agricultural, water, and soil), research samples generally, purified samples generally, and lysed cells.

7. A method for genotyping nucleic acid from a sample comprising:
a) amplifying the nucleic acid with a plurality of amplification primer pairs to form a plurality of amplification products; wherein at least one of each of said primer pairs comprises one of at least five different labels; wherein each of said amplification products comprise a different STR marker yielding an STR marker amplification product;
b) separating each of the STR marker amplification products; wherein
i) a first primer set labeled with a first label comprises at least three STR marker amplification products selected from D3S1358, vWA, TPOX, D7S820, D10S1248, and D2S441;
ii) a second primer set labeled with a second label comprises at least three STR marker amplification products selected from D5S818, vWA, D21S11, TH01, D19S433, SE33, D2S1338, and D18S51 and a sex-determination marker AMEL;
iii) a third primer set labeled with a third label comprises at least three STR marker amplification products selected from D2S441, D19S433, D3S1358, TH01, D22S1045, vWA, and FGA;
iv) a fourth primer set labeled with a fourth primer set comprises at least three STR marker amplification products selected from D22S1045, D8S1179, D13S317, D16S539, D1S1656, CSF1PO, and D2S1338; and
v) a second primer set labeled with a fifth primer set comprises at least three STR marker amplification products selected from D10S1248, D1S1656, D16S539, D12S391, D2S1338 and CSF1PO.

8. The method of claim 7, wherein at least four label channels comprises at least four different STR marker amplification products.

9. The method of claim 8, wherein D5S818 in the second primer set can also be so labeled such that it can be substituted for D8S1179 in the fourth primer set and D8S1179 can be so labeled such that it can be substituted for D5S818 in the second label channel.

10. The method of claim 8, wherein D7S820 in the first primer set can also be so labeled such that it can be substituted for D21S11 in the second primer set or CSF1PO in the fifth primer set and D21S11 can be so labeled such that it can be substituted for D7S820 in the first primer set or CSF1PO in the fifth primer set and CSF1PO can be so labeled such that it can be substituted for D7S820 in the first primer set or D21S11 in the second label channel.

11. The method of claim 7, wherein the nucleic acid is DNA, RNA or cDNA.

12. The method of claim 7, wherein the sample is selected from whole blood, a tissue biopsy, lymph, bone, bone marrow, tooth, skin, for example skin cells contained in fingerprints, bone, tooth, amniotic fluid containing placental cells, and amniotic fluid containing fetal cells. hair, skin, semen, anal secretions, feces, urine, vaginal secretions, perspiration, saliva, buccal swabs, various environmental samples (for example, agricultural, water, and soil), research samples generally, purified samples generally, and lysed cells.

13. A method of simultaneously determining the alleles present in at least four STR loci from one or more DNA samples, comprising:
a) selecting a set of at least four STR loci of the DNA sample to be analyzed which can be amplified together, wherein the at least four loci in the set are selected from the group of loci consisting of:
an InDel, SE33, D5S818, D7S820, D16S539, D18S51, D19S433, D21S11, D2S1338, D3S1358, D8S1179, FGA, TH01, VWA, TPOX, D13S317, CSF1PO, D10S1248, D12S391, D1S1656, D22S1045, D6S1043, D2S1360, D3S1744, D4S2366, D5S2500, D6S474, D6S1043, D8S1132, D7S1517, D10S2325, D21S2055, D10S2325, D2S441, D10S1248, Penta E, Penta D, LPL, F13B, FESFPS, F13A01, Penta C, DYS391, D12S391, AMEL, DYS19, DYS385, DYS389-I DYS389-II, DYS390, DYS392, DYS393, DYS437, DYS438, DYS439, and SPY;
b) co-amplifying the loci in the set in a multiplex amplification reaction, wherein the product of the reaction is a mixture of amplified alleles from each of the co-amplified loci in the set; and
c) evaluating the amplified alleles in the mixture to determine the alleles present at each of the loci analyzed in the set within the DNA sample.

14. The method of claim 13, wherein the InDel is rs2032678.

15. The method of claim 13, wherein at least four label channels comprises at least four different STR marker amplification products.

16. The method of claim 13, wherein the set of at least four loci co-amplified therein is a set of four loci, wherein the set of four loci is selected from the group of sets of loci consisting of: SE33, D5S818, D7S820, AMEL;
SE33, D22S1045, AMEL, DYS391;
SE33, Penta E, DYS391, AMEL;
SE33, D12S391, DYS391, AMEL; and
D12S391, D2S13600, AMEL, SE33.

17. The method of claim 13, wherein D7S820 in the first primer set can also be so labeled such that it can be substituted for D21S11 in the second primer set or CSF1PO in the fifth primer set and D21S11 can be so labeled such that it can be substituted for D7S820 in the first primer set or CSF1PO in the fifth primer set and CSF1PO can be so labeled such that it can be substituted for D7S820 in the first primer set or D21S11 in the second label channel.

18. A kit comprising oligonucleotide primers for co-amplifying a set of loci of at least one DNA sample to be analyzed; wherein the set of loci can be co-amplified; wherein the primers are in one or more containers; and wherein the set of loci comprises the Amelogenin locus, the STR loci D16S539, D18S51, D19S433, D21S11, D3S1358, D8S1179, FGA TH01, VWA, TPOX, DS818, D7S820, D13S317, CSF1PO, and at least one or more of the group consisting of the STR loci D2S1338, D10S1248, D12S391, D1S1656, D22S1045, D6S1043, SE33, Penta D, Penta E, D2S1360, D3S1744, D4S2366, D5S2500, D6S474, D8S1132, D7S1517, D1052325, D2152055, D22S1045, D2152055, D6S1043, D2S441, DYS19, DYS385, DYS389-I DYS389-II, DYS390, DYS392, DYS393, DYS437, DYS438, DYS439, and the SPY locus.

19. The kit of claim 18, wherein all of the oligonucleotide primers in the kit are in one container.

20. The kit of claim 18, further comprising at least one of: reagents for at least one multiplex amplification reaction, a container having at least one size standard, wherein the size standard is selected from a DNA marker and a locus-specific allelic ladder.

21.-22. (canceled)
